(19) United States
(12) Patent Application Publication
(10) Pub. No.: US 2011/0166635 A1
(43) Pub. Date: Jul. 7, 2011

(54) Therapeutic pad holder

(76) Inventor: Daniel E. Nelson, Osceola, WI (US)

(21) Appl. No.: 11/726,496

(22) Filed: Mar. 23, 2007

Related U.S. Application Data
(60) Provisional application No. 60/788,939, filed on Apr. 4, 2006.

(51) Int. Cl. A61F 7/08; A61F 7/10

(52) U.S. Cl. 607/112

(57) ABSTRACT

A therapeutic pad holder has a flexible body with a pocket for holding a hot pack, cold pack or cushion. Releasable fasteners on the body and pads separated from the body with elastic members cooperate to retain the holder around a cylindrical member or part of the human body.

### CROSS REFERENCE TO RELATED APPLICATION

This application claims the priority benefit of U.S. Provisional Patent Application Ser. No. 60/788,939 filed Apr. 4, 2006.

### FIELD OF THE INVENTION

The invention is in the field of therapeutic pad holders for supporting cushions and hot or cold packs in close proximity to human body parts.

### BACKGROUND OF THE INVENTION

Many devices are known for applying localized pressure, heat, or cold to the human anatomy. The conventional heating pad and ice bag are two examples of such prior art. While these and other similar devices meet their functional goal of heating or cooling localize portions of the body, these devices generally lack any provision for easy, quick, and secure attachment to the body portion being treated. The conventional ice bag, for example, must be manually held in place, either by the person undergoing treatment or by a nurse or aid attending the patient. Various attempts have been made to provide body wraps or bands for the specific purpose of holding in place a pressure and a hot or cold pack such as an ice bag or the like. These prior art efforts typically employ various kinds of elastic bandage-like structures for holding the heat transfer pack against the body. Examples of a human body wrap and warmer accommodating hot or cold packs are disclosed by H. E. Abare in U.S. Pat. No. 4,527,566 and W. T. Trzeciak in U.S. Pat. No. 6,427,678.

Ice fishing in winter is a popular outdoor activity in Northern United States and Canada. Walking onto a frozen lake, drilling hole in the ice, and sitting on a bucket or chair with a jig stick in hand in temperatures below freezing is ice fishing, a therapeutic experience. Out on the ice the hassles of work, the to-do lists, taxes, wars and politicians all fade away into the background. A field of ice is a landscape that sears the sin from the soul. Parkas, insulated pants and boots and bulking gloves are the ice fishing attire to maintain at least some body heat. Hot packs in parka pockets are used to add heat and prevent frostbite. Gloves and mittens inhibit ice fisherpersons from using a finger on the fish line to sense a bite, baiting a hook with a minnow and removing a fish hook from a fish. The gloves and mittens must be removed from the fisherperson's hand to accomplish these tasks. In sub-zero temperatures on the ice bare hands are subject to rapid frostbite.

### SUMMARY OF THE INVENTION

The invention is a therapeutic pad holder for accommodating hot packs, cold packs and cushion pads to alleviate discomfort and cold human body parts. The pad holder has a generally flat body having a pocket for holding hot and cold packs or cushion pads located between sheet members. An opening into the pocket is closed with a movable flap which can be located in a position to allow the hot and cold packs and cushion pads to be inserted into and removed from the pocket. Releasable fasteners, such as Velcro hook and loop pads having hook and loop elements are used to retain the pad holder around a person's body part or around a rod, such as a ice fishing rod. Elastic members secured to the body support members hold one of the Velcro pads. The other Velcro pads are secured to the body. The holder is useable to support and apply pressure to human body parts, including but not limited to arms, wrists, knees, shins and feet.

### DESCRIPTION OF THE DRAWING

FIG. 1 is a perspective view of the therapeutic pad holder of the invention;

FIG. 2 is an enlarged front elevational view of FIG. 1;

FIG. 3 is an enlarged rear elevational view of FIG. 1;

FIG. 4 is a top plan view of FIG. 2;

FIG. 5 is a bottom plan view of FIG. 2;

FIG. 6 is a side elevational view of the right side of FIG. 2;

FIG. 7 is a side elevational view of the left side of FIG. 2;

FIG. 8 is an enlarged sectional view taken along line 8-8 of FIG. 2;

FIG. 9 is an enlarged sectional view taken along line 9-9 of FIG. 2; and

FIG. 10 is a diagrammatic view of an ice fisherperson using the therapeutic pad holder of the invention.

### DESCRIPTION OF INVENTION

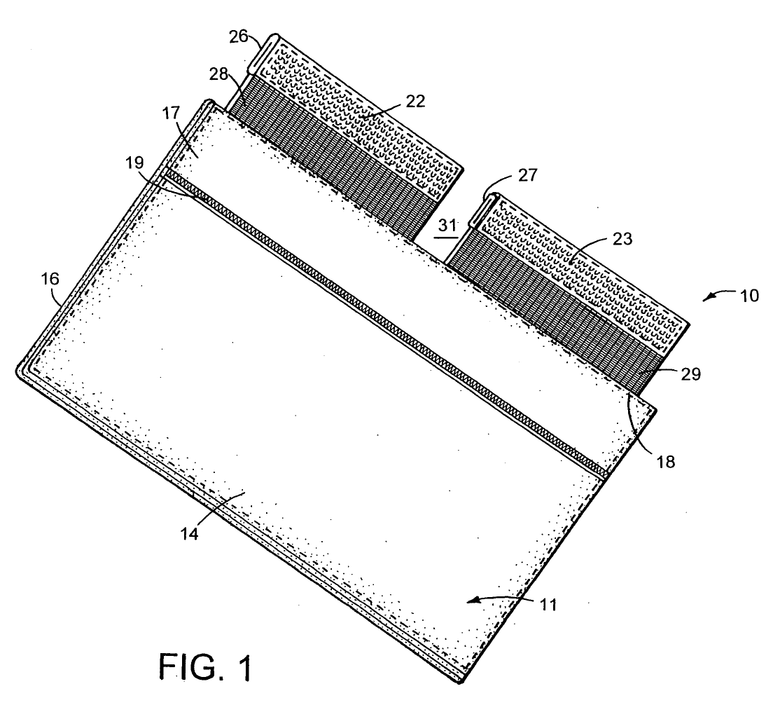

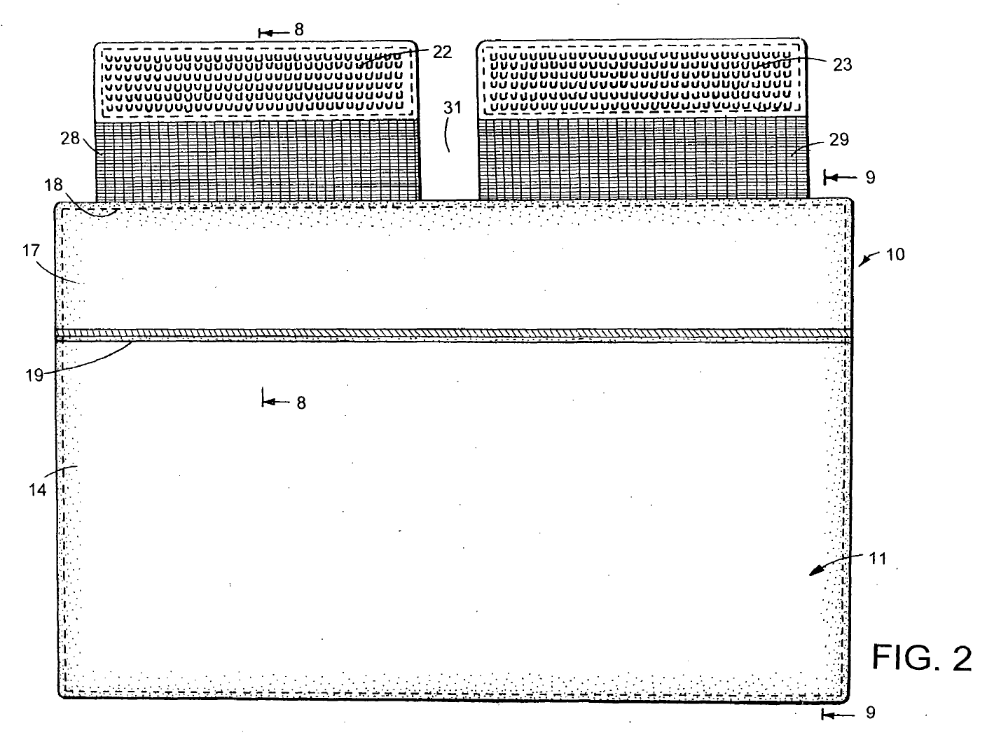

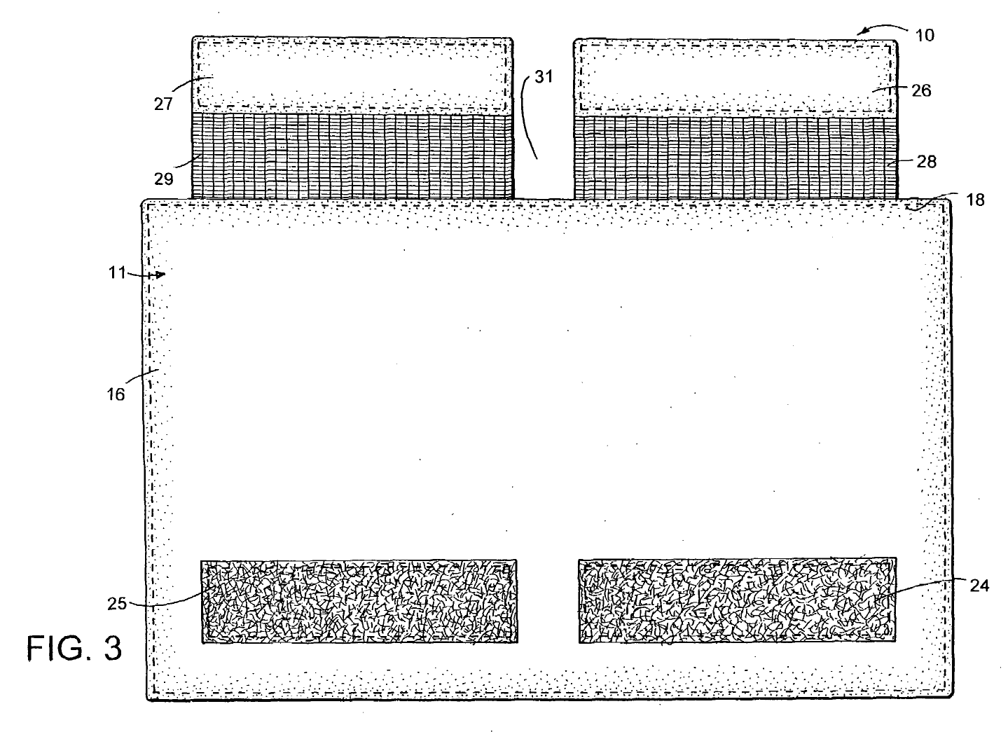

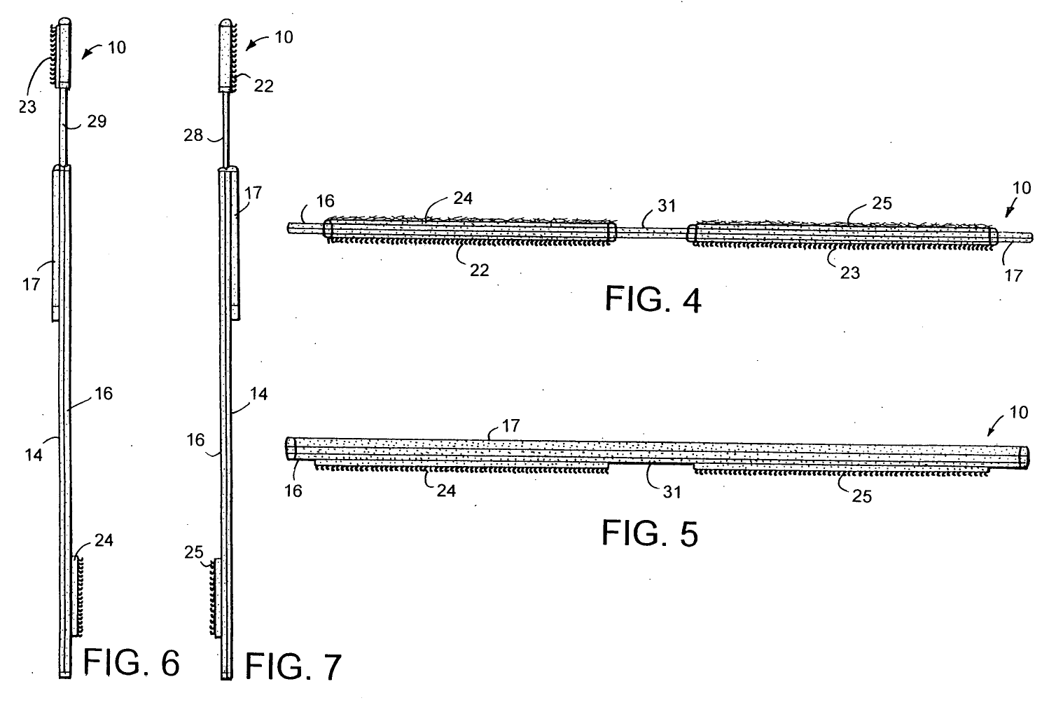

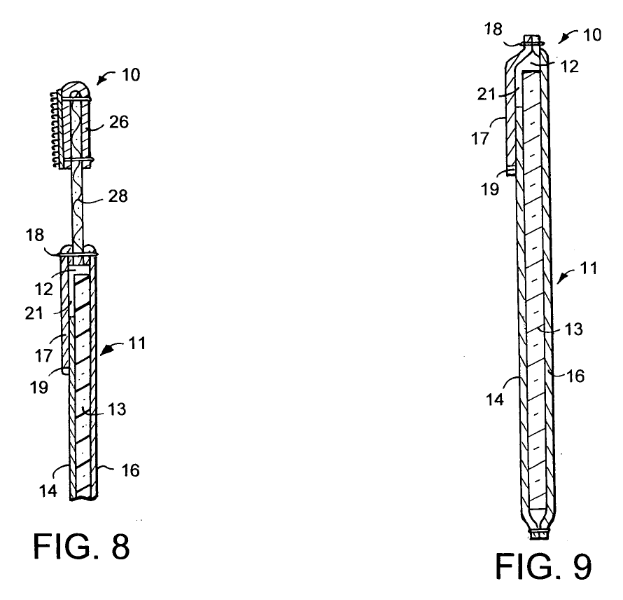

The therapeutic pad holder 10, shown in FIGS. 1-3, 8 and 9, has a generally rectangular body 11 surrounding a pocket 12 for accommodating a pad 13. Body 11 has a width which can be wrapped around a rod or a part of a person's body, such as an ankle, wrist or elbow. Pad 13 is a flexible plastic open cell plastic cushion used to protect the person's body part and keep the body part warm. Pad 13 can be a hot pack to alleviate discomfort caused by muscular strain or ambient cold temperatures. An example of a hot pack is a flexible waterproof pouch containing ingredients that generate heat through a chemical reaction that occurs by exposing the ingredients of iron, water, cellulose, vermiculite, activated carbon and salt, is marketed by Grabber Warmers of Grand Rapids, Mich. The hot pack has material that retains its shape in use in pad holder 10. Alternatively, pad 13 can be an ice pack used to reduce swelling associated with bruises or various types of surgery. The pad can have different dimensions, flexibility and hardness.

Pad holder 10 has a rectangular front sheet member 14 stitched to a rectangular back sheet member 16 with pocket 12 located between sheet members 14 and 16. Front sheet member 14 is a polyester flexible and air pervious fabric that does not absorb water. The back sheet member 16 is a flexible water repellant Nylon fabric. As shown in FIGS. 1, 2, 8 and 9, an elongated flap 17 is secured with stitches 18 to the top horizontal edge of sheet member 16. Flap 17 is a polyester fabric. Other types of materials and fabrics can be used for sheet members 14 and 16 and flap 17. The bottom longitudinal edge 19 of flap 17 is cross stitched to provide a strong non-tear edge. Flap 17 overlaps an upper linear portion of front sheet member 14 and covers an opening 21 or inlet to pocket 12. Flap 17 can be folded away from sheet member 14 to provide access to opening 21 and pocket 12 and allow insertion into and removal of pad 13 from pocket 12.

Body 11 is retained around a human body part or rod with releasable fasteners comprising a pair of hook-type pads 22 and 23 cooperating with a pair of loop-type pads 24 and 25, such as Velcro releasable fasteners. Pads 22 and 23 are attached with an adhesive to inverted U-shaped bands 26 and 27 stitched to rectangular elastic members 28 and 29. The bottom linear edges of elastic members 28 and 29 are secured with stitches 18 to back sheet member 16 and flap 17. Elastic members 28 and 29 have the same widths and lengths and are separated with a space 31. The elastic members 28 and 29 have width elasticity allowing the width dimension to increase to fit the human body part and bias holder 10 around the body part. The members 28 and 29 are relatively inelastic in longitudinal directions to impede holder 10 from walking or moving along the human body part. The number of elastic members and releasable fasteners can be increased along with the size of the body 11.

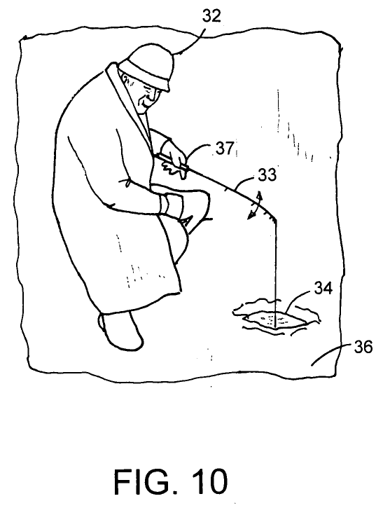

An ice fisherperson 32, shown in FIG. 10, is using a jig stick 33 to retain a fish line and bobber in an open ice fishing hole 34 in ice 36 on Lake Bemidji, Minn. in sub-zero temperature. A therapeutic pad holder 37 containing a hot pack is located around the handle end of the jig stick 33 to keep the fisherperson's hand warm. Additional therapeutic pad holders may be used to keep the arms, feet and legs of the fisherperson warm.

The overall size of therapeutic pad holder 10 enables the holder to be used for applying either heat or cold to relatively large body portions such as the back or chest of a person. The size or number of pockets in the pad holder, or in any pad holder according to the present invention, are considered to be matters of choice, depending primarily on the particular application for which the pad holder is intended. In that regard, it should now be understood that pad holders of various lengths and widths can be readily prepared for fitting specific body portions such as the jaw, the knee, the shin, the shoulder, and others in addition to the body members described above. Ribs and stays can be incorporated in the sheet members to retain the holder in a selected curved configuration.

The present invention is adaptable for holders intended to cover portions of the body, such as an individual hand or foot. The holder can be located around a person's wrist to relieve stress or pressure incurred at the hand or wrist that could telescope up the forearm. The wrist support eases discomfort caused by carpal tunnel syndrome. The holder when attached to a person's leg can function as a shin guard or an ankle support.

It should also be understood that the foregoing relates only to specific embodiments of the present invention, and that numerous uses, changes and modifications may be made therein without departing from the spirit and scope of the invention.

1. A holder for a pad comprising: a body having a front sheet member and a back sheet member, a pocket for accommodating a pad located between said front and back sheet members, said front and back sheet members having adjacent opposite side, bottom and top edges, first fasteners securing the opposite sides and bottom edges together around said pocket, said adjacent top edges being separated to provide an opening into said pocket, a flap having opposite sides and top edges, said flap extended over the opening into said pocket and a top portion of the front sheet member for retaining a pad in said pocket, second fasteners securing the opposite side edges of the front sheet member and the top edge of the back sheet member to the opposite sides and top edges of the flap to retain the flap adjacent the top portion of the front sheet, a pair of hook members spaced from the top edge of the back sheet member, a pair of laterally spaced elastic members securing the hook members to said top edge of the back sheet member, and a pair of loop members secured to the back member adjacent the bottom edge thereof, said hook members being releasable engageable with said loop members to retain said holder around a generally cylindrical member.

2. The holder of claim 1 wherein: said front and back sheet members are flexible generally rectangular fabric sheet members.

3. The holder of claim 2 wherein: said flap is a flexible generally rectangular fabric sheet member.

4. The holder of claim 1 wherein: each hook member is a generally rectangular pad having hook elements joined to an extended away from the pad in the direction of the front sheet member and each loop member is a generally rectangular pad having loop elements joined to and extended away from the rear sheet member, said hook elements and loop elements cooperating with each other to releasably connect the hook and loop members.

5. The holder of claim 1 wherein: each of said elastic members are generally rectangular elastic sheets that allow the hook members to be moved away from the top edges of the back sheet member whereby the elastic sheets bias the holder around the cylindrical member.

6. The holder of claim 1 wherein: the front sheet member is a polyester flexible air pervious fabric, and the back sheet member is a flexible water repellant Nylon fabric.

7. The holder of claim 1 including: a pad comprising an open cell plastic cushion located in said pocket.

8. The holder of claim 1 including: a pad comprising a hot pack located in said pocket.

9. The holder of claim 1 including: a pad comprising a cold pack located in said pocket.

10. A holder for a pad comprising: a body having a front member and a back member, a pocket for accommodating a pad located between said front and back members, said front and back members having adjacent opposite sides, bottom and top edges, first fasteners securing the adjacent opposite sides and bottom edges together around said pocket, said adjacent top edges being separated to provide an opening into said pocket, a flap having opposite sides and top edges, said flap extended over the opening into said pocket and an upper portion of the front member for retaining a pad in said pocket, second fasteners securing the opposite sides of the front member and the top edge of the back member to the opposite sides and top edges of the flap to retain the flap adjacent the top portion of the front member, a first member spaced from the top edges of the front and back members, an elastic member securing the first member to the top edge of the back member and releasable fasteners on the first member and one of the front or back members to retain said holder around a separate member.

11. The holder of claim 10 wherein: said releasable fasteners are hook and loop releasable elements.

12. The holder of claim 10 including: a second member laterally spaced from the first member and spaced from the top edges of the front and back members, and an elastic member securing the second member to one of the top edges of the front and back members, and releasable fasteners on the second member and one of the front and back members to further retain the holder around the separate member.

13. The holder of claim 12 wherein: the elastic members are elastic sheets that allow the first and second members to be moved away from the top edges of the first and second members whereby the elastic sheets bias the holder around the separate member.

14. The holder of claim 10 wherein: said front and back members are flexible fabric sheet members.

15. The holder of claim 10 wherein: the front member is a polyester flexible air pervious fabric, and the back member is a flexible water repellant Nylon fabric.

16. The holder of claim 10 including: a pad comprising an open cell plastic cushion located in said pocket.

17. The holder of claim 10 including: a pad comprising a hot pack located in said pocket.

18. The holder of claim 10 including: a pad comprising a cold pack located in said pocket.
